(19) United States
(12) Patent Application Publication
(10) Pub. No.: US 2025/0354104 A1
(43) Pub. Date: Nov. 20, 2025

(54) Single colony multi-stage coating-separating device for high-pressure environment and control method

(71) Applicant: Guangdong University of Technology, Guangzhou (CN)

(72) Inventors: Jingchun FENG, Guangzhou (CN); Si ZHANG, Guangzhou (CN); Song ZHONG, Guangzhou (CN); Yi WANG, Guangzhou (CN); Zhifeng YANG, Guangzhou (CN)

(73) Assignee: Guangdong University of Technology, Guangzhou (CN)

(21) Appl. No.: 18/664,288

(22) Filed: May 15, 2024

(51) Int. Cl. C12M 1/26; C12M 1/36

(52) U.S. Cl. CPC C12M 33/00; C12M 41/48

(57) ABSTRACT

A single colony multi-stage coating-separating device for a high-pressure environment is provided, including a culture device and a liquid injection port installed at the top of an inner wall of the culture device. At least two culture dishes are stacked at intervals in the culture device along an axial direction of the culture device, the center of each culture dish is provided with an avoiding hole, and the avoiding hole radially expands towards an edge of the culture dish to form a fan-shaped separating opening. Adjacent separating openings are staggered in the vertical direction, a stirring rod is arranged to penetrate the central axis of the culture device, a lower end of the stirring rod is sleeved with a coating unit, and the projection of the coating unit on the horizontal plane falls within the projection of the separating openings.

### TECHNICAL FIELD

The present invention relates to the technical field of microorganism culturing, and more particularly to a single colony multi-stage coating-separating device for a high-pressure environment and a control method.

### BACKGROUND

In the prior art, the separation of microorganisms in special marine habitats is mainly carried out in an atmospheric pressure environment. It is rare to separate and cultivate single colonies rarely in a high-pressure environment. Even if the single colonies are separated in the high-pressure environment, its separation efficiency is low and the operation process is complicated, resulting in the number of microorganisms that can be cultivated at present being less than 1% of that in the deep-sea environment, which brings us difficulties in correctly understanding and utilizing the marine resources. Rapid and widely effective techniques and methods for separating deep-sea microorganisms are significant means that restrict the efficient utilization of deep-sea microbial resources.

The patent application CN114456909A discloses a separating and culturing device for deep-sea microorganism. By providing internal threads on an inner wall surface of a base, a sample of a cultured bacteria solution is introduced into the internal threads of the separating and culturing device through an inlet pipeline. Under the action of gravity, the sample slides down along the internal threads, and during the movement, the microbial bacteria solution carried by the sample will gradually decrease, achieving the separation of microorganisms. Thus, this realizes automatic streaking separation of the environmentally sampled and enriched microbial bacteria solution under in situ marine environmental conditions, effectively improving the efficiency of solid separation and culture of microorganisms, and providing an important fundamental means for the separation and culture of efficient marine special microbial bacteria.

However, the above device relies solely on the action of gravity for sample separation, and can only perform a single streaking separation, which greatly reduces the probability for single colonies being separated.

### SUMMARY

For the above problem, the present invention provides a single colony multi-stage coating-separating device for a high-pressure environment and a control method, which mainly intend to solve the problems that existing microorganism high-pressure separating devices are not applicable for a sample coated uniformly in a large area and it is difficult to separate single colonies.

To solve the above technical problems, a first aspect of the present invention provides a single colony multi-stage coating-separating device for a high-pressure environment, which includes a culture device and a liquid injection port installed at the top of an inner wall of the culture device. At least two culture dishes are stacked at intervals in the culture device along an axial direction of the culture device, the center of each culture dish is provided with an avoiding hole, the avoiding hole radially expands towards an edge of the culture dish to form a fan-shaped separating opening, adjacent separating openings are staggered in the vertical direction, a stirring rod is arranged to penetrate the central axis of the culture device, there is no motion interference between the stirring rod and the avoiding holes, a lower end of the stirring rod is sleeved with a coating unit, and the projection of the coating unit on the horizontal plane falls within the projection of the separating openings.

In some embodiments, a baffle is also included, and the baffle is installed on the left side of each separating opening.

In some embodiments, the coating unit includes a sleeve slidably connected to the stirring rod and a connecting rod fixed to an outer side of the sleeve, wherein the other end of the connecting rod is installed with a scratching board.

In some embodiments, a cross-section of the stirring rod is rectangular, the sleeve and the stirring rod are in clearance fit, the avoiding hole is circular, and a diameter of the avoiding hole is larger than or equal to a diagonal length of the sleeve.

In some embodiments, the top of the culture device is provided with a removable cover, and the bottom of the culture device is provided with a leg.

In some embodiments, the culture dishes are removable.

In some embodiments, the edge of the culture dish is provided with a first buckle, and the inner wall of the culture device is provided with a second buckle that cooperates with the first buckle.

In some embodiments, an angle between two of the adjacent separating openings is less than or equal to an angle at which the separating opening expands.

In some embodiments, the distance between two of the adjacent culture dishes is greater than the height of the coating unit.

A second aspect of the present invention provides a control method for the single colony multi-stage coating-separating device for a high-pressure environment as described above, comprising: loading solid culture media into the culture dishes, wherein the height of the solid culture medium is flush with the upper end surface of the corresponding culture dish; disposing the coating unit on the surface of the uppermost culture dish; and driving the stirring rod according to a preset rotation speed and a preset time.

The beneficial effects of the present invention are as follows, by vertically arranging the multi-stage culture dishes in the culture device, each culture dish being provided with the separating opening therein, and using the stirring rod to drive the coating unit to perform rotational movement, when the coating unit moves to the separating opening area of a current level, the coating unit loses the support provided by the corresponding culture dish, and automatically falls onto the culture dish of a next level under the action of gravity, thereby continuing to be driven by the stirring rod to perform coating. During the rotation of the stirring rod, since the center of the culture dish is provided with the avoiding hole, the stirring rod will not drive the culture dish to rotate.

### BRIEF DESCRIPTION OF THE DRAWINGS

FIG. 1 is a schematic structural diagram of a single colony multi-stage coating-separating device for a high-pressure environment disclosed in Embodiment 1 of the present invention;

FIG. 2 is a schematic three-dimensional structural diagram of a culture dish disclosed in Embodiment 1 of the present invention;

FIG. 3 is a schematic diagram of a top view structure of the culture dish disclosed in Embodiment 1 of the present invention;

FIG. 4 is a schematic three-dimensional structural diagram of a coating unit disclosed in Embodiment 1 of the present invention; and

FIG. 5 is a schematic sectional structural diagram of the coating unit disclosed in Embodiment 1 of the present invention.

### DETAILED DESCRIPTION OF THE EMBODIMENTS

In order to make the objectives, technical solutions, and advantages of the present invention clearer and more definite, the content of the present invention is further explained in detail below in conjunction with the accompanying drawings and specific embodiments. It should be understood that the specific embodiments described herein are only intended to explain the present invention and are not intended to limit the present invention. Additionally, it should be noted that for ease of description, only parts related to the present invention, and not all contents, are shown in the accompanying drawings.

### Embodiment 1

The present embodiment provides a single colony multi-stage coating-separating device for a high-pressure environment, which separates and cultivates microorganisms by reshaping their in-situ environment to perform automatic multi-stage coating. The device and method enhances the culturability of marine microorganisms and provides an important foundational means for the development and utilization of high-pressure environment microbial resources.

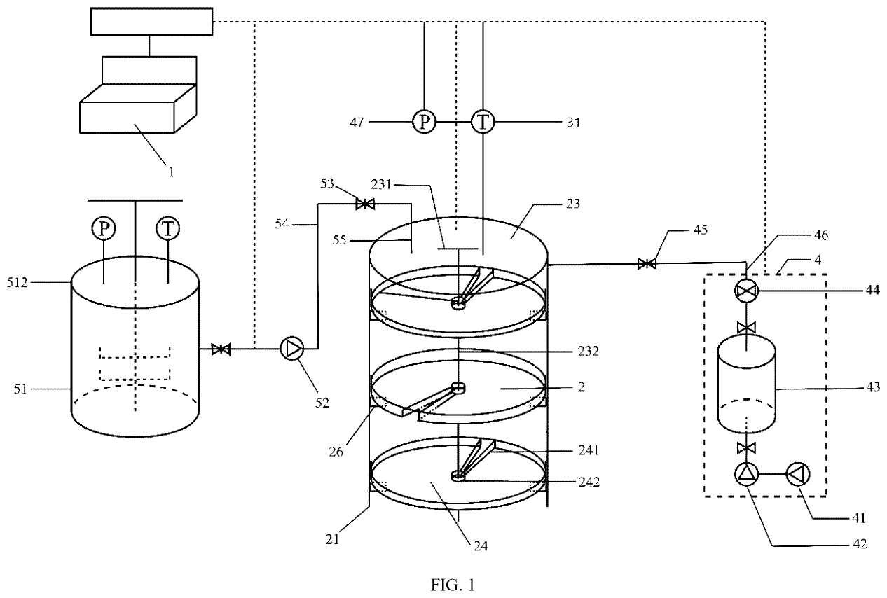

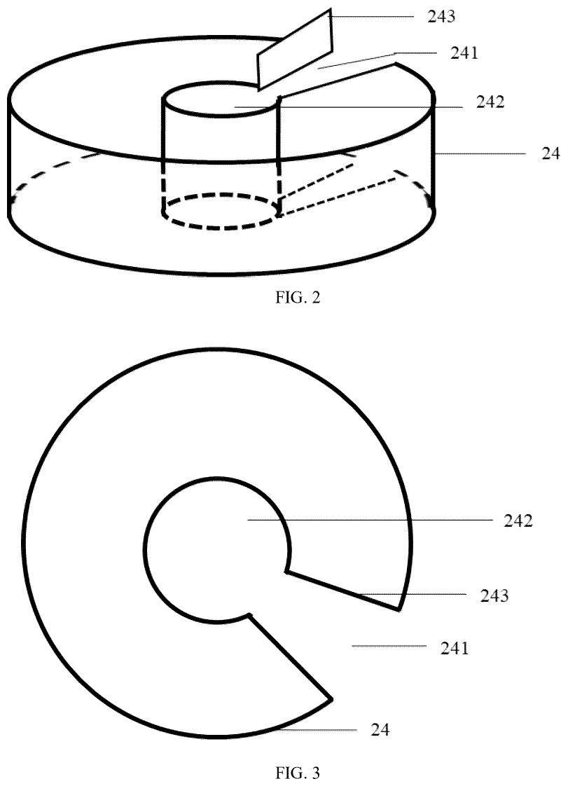

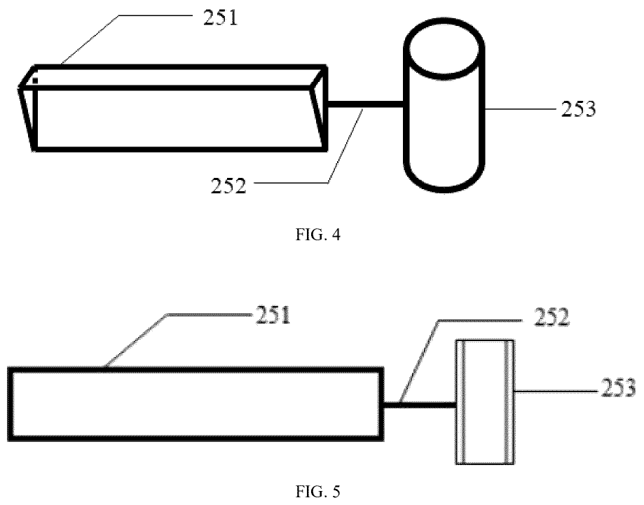

As shown in FIGS. 1-5, the device mainly includes a culture device 2 and a liquid injection port 55 installed at the top of an inner wall of the culture device 2. At least two culture dishes 24 are stacked at intervals in the culture device along an axial direction of the culture device 2, the center of each culture dish 24 is provided with an avoiding hole 242, the avoiding hole 242 radially expands towards an edge of the culture dish 24 to form a fan-shaped separating opening 241, adjacent separating openings 241 are staggered in the vertical direction, a stirring rod 232 is arranged to penetrate the central axis of the culture device 2, there is no motion interference between the stirring rod 232 and the avoiding holes 242, a lower end of the stirring rod 232 is sleeved with a coating unit 25, and the projection of the coating unit 25 on the horizontal plane falls within the projection of the separating openings 241.

In the present embodiment, by vertically providing the multi-stage culture dishes 24 in the culture device 2, each culture dish 24 being provided with the separating opening 241 therein, and using the stirring rod 232 to drive the coating unit 25 to perform rotational movement on the surface of the culture medium of the culture dish 24, when the coating unit 25 moves to an area of the separating opening 241 of the culture dish 24 of a current level, the coating unit 25 loses the support provided by the culture dish 24, and automatically falls onto the surface of the culture dish 24 of a next level under the action of gravity, thereby continuing to be driven by the stirring rod 232 to perform coating. During the rotation of the stirring rod 232, since the center of the culture dish 24 is provided with the avoiding hole 242, the stirring rod 232 will not drive the culture dish 24 to rotate.

In a preferred implementation, a baffle 243 is also included, and the baffle 243 is installed on the left side of the separating opening 242. In this implementation, the main function of the baffle 243 is to block a bacterial solution dripping from the liquid injection port 55, preventing it from spreading into the next level on its own. Meanwhile, the baffle 243 enables the coating unit 25 to smoothly fall onto the surface of the culture medium in the culture dish 24 of a next level, so that the stirring rod 232 continues driving the coating unit 25 to perform coating, thereby ensuring the effectiveness of coating. Specifically, the coating unit 25 should be positioned on the left side of the baffle 243, and the vertical direction of the liquid injection port 55 should be located in the center of the left side of the baffle 243, so that the bacterial solution can drip down onto the culture medium loaded in the culture dish 24, and the coating unit 25 can coat immediately. Predictably, the bacterial solution dripping from the liquid injection port 55 will converge on the left side of the baffle 243. Due to the blocking effect of the baffle 243, the bacterial solution can only enter the next level through the separating opening 241 of the current level after the bacterial solution passes around the culture dish 24 for one circle by means of the coating unit 25.

The aforementioned coating unit 25 may be any mechanical device that can be driven by the stirring rod 232 to rotate and slide axially along the stirring rod 232. In one example, the coating unit 25 includes a sleeve 253 slidably connected to the stirring rod 232, and a connecting rod 252 fixed to the outer wall of the sleeve 253, wherein the other end of the connecting rod 252 is installed with a scratching board 251. The cross-section of the stirring rod 232 is rectangular, and the sleeve 253 is in clearance fit with the stirring rod 232. The avoiding hole 242 is circular, and its diameter is greater than or equal to the diagonal length of the sleeve 253, ensuring that there is no motion interference between the stirring rod 232 and the avoiding hole 242. In the above solution, the stirring rod 232 and the sleeve 253 are provided to be square in shape, thereby enabling the transmission of torque from the stirring rod 232 to the sleeve 253, and then transmitting the torque to the connecting rod 252 and the scratching board 251 through the sleeve 253. Meanwhile, the square fit also allows the sleeve 253 to slide along the outer wall of the stirring rod 232. Therefore, under the action of gravity, the sleeve 253 can carry the connecting rod 252 and the scratching board 251 to drop vertically, functioning as the mechanical basis for the coating unit 25 to automatically fall into the culture dish 24 of a next level.

Optionally, the top of the culture device 2 is provided with a removable cover 23, and the bottom of the culture device 2 is provided with legs 21. In this solution, the cover 23 can be connected to the culture device 2 via buckles. The liquid injection port 55, a temperature sensor 31, a pressure sensor 47, and the stirring rod 232 may be fixed to the cover 23. The legs 21 are intended to elevate the placement height of the culture device 2, leaving space for the bottom end of the stirring rod 232.

Furthermore, each culture dish 24 is designed to be removable, which facilitates the disassembling of the culture dish 24 from the inner wall of the culture device 2. In one example, the edge of the culture dish 24 is provided with a first buckle, and the inner wall of the culture device 2 is provided with a second buckle that cooperates with the first buckle.

The culture dishes 24 are filled with solid culture media of different nutritional ratios of culture media. The present invention involves three culture dishes 24, i.e., the first culture dish, the second culture dish, and the third culture dish. The number of the culture dishes 24 may be determined according to experimental needs and is not limited herein. The angle between two adjacent separating openings 241 is less than or equal to the angles at which the separating openings 241 expand; that is, in a vertical projection perspective, the separating opening 241 of the culture dish 24 of a next lower level should be staggered from the separating opening 241 of the culture dish 24 of a current level, and the staggering angle should be less than or equal to the angle at which the separating opening 241 expands, so as to avoid reducing a coatable area of the culture dish 24 of the next lower level.

To ensure the smooth rotation of the coating unit 25, the distance between the two adjacent culture dishes 24 should be greater than the height of the coating unit 25, and the height of the solid culture medium should be flush with the corresponding culture dish 24.

Continuing to reference to FIG. 1 for further description of the auxiliary devices of the present device, a pressure control unit 4 is also included. The pressure control unit 4 is mainly used to inject gas into the culture device 2 to increase the pressure, allowing the pressure environment inside the culture device 2 to be consistent with the pressure value encountered by microorganisms in their in-situ marine environment, while monitoring changes in pressure inside the culture device 2. The pressure control unit 4 includes an air compressor 41, a booster pump 42, a gas storage tank 43, a pressure regulating valve 44, and a gas delivery pipeline 46 connected in sequence. The middle of the gas delivery pipeline 46 is provided with a valve 45, and the tail end of the gas delivery pipeline 46 is directly connected to the cover 23 or the side surface of the culture device 2. An enrichment system 5 is also included, and includes a microbial enrichment kettle 51, a micro-injection pump 52, and a liquid outlet pipeline 54 connected in sequence. To ensure the stable pressure of the entire device, the middle of the outlet pipeline 54 is provided with a valve 53, and the tail end of the outlet pipeline 54 enters the interior of the culture device 2 from the cover 23, and forms the aforementioned liquid injection port 55. The temperature sensor 31, the pressure sensor 47, the pressure control unit 4, and the stirring rod 232 described above all acquire data by a control unit 1 and are operated and controlled by the control unit 1.

### Embodiment 2

The present embodiment provides a control method for the single colony multi-stage coating-separating device for a high-pressure environment as described above, comprising: loading solid culture media into the culture dishes 24, the height of each solid culture medium being flush with the upper end surface of the corresponding culture dish 24; disposing the coating unit 25 on the surface of the uppermost culture dish 24; and driving the stirring rod 232 according to a preset rotation speed and a preset time. In the present embodiment, with reference to the way of setting the baffle 243 described in Embodiment 1, a motor should drive the stirring rod 232 to rotate counterclockwise.

The above embodiments are only intended to illustrate the technical concept and characteristics of the present invention, and their purpose is to enable those of ordinary skills in the art to understand the content of the present invention and implement it accordingly, and not to limit the scope of the present invention. All equivalent variations or modifications made according to the essence of the content of the present invention should be covered by the scope of the present invention.

What is claimed is:

1. A single colony multi-stage coating-separating device for a high-pressure environment, comprising a culture device and a liquid injection port installed at a top of an inner wall of the culture device, wherein
at least two culture dishes are stacked at intervals in the culture device along an axial direction of the culture device, a center of each culture dish of the at least two culture dishes is provided with an avoiding hole, the avoiding hole radially expands towards an edge of the each culture dish to form a fan-shaped separating opening, adjacent fan-shaped separating openings are staggered in a vertical direction, a stirring rod is arranged to penetrate a central axis of the culture device, there is no motion interference between the stirring rod and the avoiding hole, a lower end of the stirring rod is sleeved with a coating unit, and a projection of the coating unit on a horizontal plane falls within a projection of the fan-shaped separating opening.

2. The single colony multi-stage coating-separating device for the high-pressure environment according to claim 1, further comprising a baffle installed on a left side of the fan-shaped separating opening.

3. The single colony multi-stage coating-separating device for the high-pressure environment according to claim 1, wherein the coating unit comprises a sleeve slidably connected to the stirring rod and a connecting rod fixed to an outer side of the sleeve, wherein a first end of the connecting rod is fixed to the outer side of the sleeve, and a second end of the connecting rod is installed with a scratching board.

4. The single colony multi-stage coating-separating device for the high-pressure environment according to claim 3, wherein a cross-section of the stirring rod is rectangular, the sleeve and the stirring rod are in a clearance fit, the avoiding hole is circular, and a diameter of the avoiding hole is larger than or equal to a diagonal length of the sleeve.

5. The single colony multi-stage coating-separating device for the high-pressure environment according to claim 1, wherein a top of the culture device is provided with a removable cover, and a bottom of the culture device is provided with a leg.

6. The single colony multi-stage coating-separating device for the high-pressure environment according to claim 1, wherein the at least two culture dishes are removable.

7. The single colony multi-stage coating-separating device for the high-pressure environment according to claim 6, wherein the edge of the each culture dish is provided with a first buckle, and the inner wall of the culture device is provided with a second buckle cooperating with the first buckle.

8. The single colony multi-stage coating-separating device for the high-pressure environment according to claim 1, wherein an angle between the adjacent fan-shaped separating openings is less than or equal to an angle formed by an expansion of the fan-shaped separating opening.

9. The single colony multi-stage coating-separating device for the high-pressure environment according to claim 1, wherein a distance between adjacent culture dishes of the at least two culture dishes is greater than a height of the coating unit.

10. A control method for the single colony multi-stage coating-separating device for the high-pressure environment according to claim 1, comprising the following steps: loading a solid culture medium into the each culture dish, a height of the solid culture medium being flush with an upper end surface of the each culture dish; disposing the coating unit on a surface of an uppermost culture dish of the at least two culture dishes; and driving the stirring rod according to a preset rotation speed and a preset time.

11. A control method for the single colony multi-stage coating-separating device for the high-pressure environment according to claim 2, comprising the following steps: loading a solid culture medium into the each culture dish, a height of the solid culture medium being flush with an upper end surface of the each culture dish; disposing the coating unit on a surface of an uppermost culture dish of the at least two culture dishes; and driving the stirring rod according to a preset rotation speed and a preset time.

12. A control method for the single colony multi-stage coating-separating device for the high-pressure environment according to claim 3, comprising the following steps: loading a solid culture medium into the each culture dish, a height of the solid culture medium being flush with an upper end surface of the each culture dish; disposing the coating unit on a surface of an uppermost culture dish of the at least two culture dishes; and driving the stirring rod according to a preset rotation speed and a preset time.

13. A control method for the single colony multi-stage coating-separating device for the high-pressure environment according to claim 4, comprising the following steps: loading a solid culture medium into the each culture dish, a height of the solid culture medium being flush with an upper end surface of the each culture dish; disposing the coating unit on a surface of an uppermost culture dish of the at least two culture dishes; and driving the stirring rod according to a preset rotation speed and a preset time.

14. A control method for the single colony multi-stage coating-separating device for the high-pressure environment according to claim 5, comprising the following steps: loading a solid culture medium into the each culture dish, a height of the solid culture medium being flush with an upper end surface of the each culture dish; disposing the coating unit on a surface of an uppermost culture dish of the at least two culture dishes; and driving the stirring rod according to a preset rotation speed and a preset time.

15. A control method for the single colony multi-stage coating-separating device for the high-pressure environment according to claim 6, comprising the following steps: loading a solid culture medium into the each culture dish, a height of the solid culture medium being flush with an upper end surface of the each culture dish; disposing the coating unit on a surface of an uppermost culture dish of the at least two culture dishes; and driving the stirring rod according to a preset rotation speed and a preset time.

16. A control method for the single colony multi-stage coating-separating device for the high-pressure environment according to claim 7, comprising the following steps: loading a solid culture medium into the each culture dish, a height of the solid culture medium being flush with an upper end surface of the each culture dish; disposing the coating unit on a surface of an uppermost culture dish of the at least two culture dishes; and driving the stirring rod according to a preset rotation speed and a preset time.

17. A control method for the single colony multi-stage coating-separating device for the high-pressure environment according to claim 8, comprising the following steps: loading a solid culture medium into the each culture dish, a height of the solid culture medium being flush with an upper end surface of the each culture dish; disposing the coating unit on a surface of an uppermost culture dish of the at least two culture dishes; and driving the stirring rod according to a preset rotation speed and a preset time.

18. A control method for the single colony multi-stage coating-separating device for the high-pressure environment according to claim 9, comprising the following steps: loading a solid culture medium into the each culture dish, a height of the solid culture medium being flush with an upper end surface of the each culture dish; disposing the coating unit on a surface of an uppermost culture dish of the at least two culture dishes; and driving the stirring rod according to a preset rotation speed and a preset time.
